(19) United States
(12) Patent Application Publication
(10) Pub. No.: US 2014/0221954 A1
(43) Pub. Date: Aug. 7, 2014

(54) Convertible Reusable Diaper

(71) Applicants: Jinmei Wang, Markham (CA); Yu Ping, Dublin, CA (US)

(72) Inventors: Jinmei Wang, Markham (CA); Yu Ping, Dublin, CA (US)

(73) Assignees: Yu Ping, Dublin, CA (US); Jinmei Wang, Markham (CA)

(21) Appl. No.: 14/245,972

(22) Filed: Apr. 4, 2014

Related U.S. Application Data
(63) Continuation-in-part of application No. 13/417,009, filed on Mar. 9, 2012, now Pat. No. 8,728,052.

(30) Foreign Application Priority Data
May 10, 2013 (CN) 201320254477.3

(51) Int. Cl. A61F 13/49

(52) U.S. Cl. CPC A61F 13/49004; A61F 2013/49098
USPC 604/385.14

(57) ABSTRACT

A convertible reusable diaper is provided, comprising an outer cover, a barrier panel portion, and at least one of a liner portion and an absorption portion. One or more first fastener members are attachable to at least one of the outer cover portion and the barrier panel portion. One or more second fastener members are attachable to at least one of the liner portion and the absorption portion, wherein at least one of the second fastener members is cooperatively engaged with at least one of the first fastener members, thereby allowing at least part of at least one of the liner portion and the absorption portion to be detachable to the outer cover portion. One or more hole-openings are formed on the barrier panel portion, thereby allowing engagement between the first and the second fastener members.

### CROSS-REFERENCE TO RELATED APPLICATION

This application is a continuation in part of Ser. No. 13/417,009, filed with the USPTO on Mar. 9, 2012, and 201320254477.3 filed with SIPO on May 10, 2013, the entire contents each of which is incorporated herein by reference in their entireties.

### TECHNICAL FIELD

The disclosure relates to diapers, and more specifically, relates to convertible reusable diapers.

### BACKGROUND

The increased ecological awareness has led to a growth in campaign for parents to turn away from disposable diaper products and choose reusable alternatives instead, such as cloth or hybrid diapers. Cloth diapers traditionally consisted of a folded square or rectangle of cloth, fastened with safety pins and required constant monitoring and changing because they quickly became soggy or soiled with feces. They must be washed after each diaper change. Although the usage of “stay-dry” wicking liner and waterproof exteriors or water-resistant outer shell in modern cloth diapers alleviates some of the need for constant changing and washing everything after each diaper change, how to make a diaper that can be adjusted, easily assembled, cleaned to accommodate different absorption and containment requirements of body discharged as well as size requirements to correspond with baby's growing weight still remains to be a challenge.

There are different types and brands of cloth diapers available on the market, including impermeable diaper covers, pocket diapers, all-in-one and all-in-two diapers with waterproof exteriors, etc. Different diapers work better for different situations and none seems to be perfect on their own. For example, at home, a pocket diaper or a diaper cover with reusable insert pads works best. However, they generally are bulky and don't work really well under most clothes, making parents reluctant to use when going out. Although all-in-one and all-in-two diapers are a bit trimmer while still providing good absorbency, they are more expensive and dry time is much longer than regular cloth diapers that go with a cover and pocket diapers, taking up more drying cycles.

Therefore, it would be desirable to provide a convertible reusable diaper product which combines the best of all the aforementioned diaper designs in the prior art and can be conveniently assembled and adjusted to correspond with different occasions by converting among different designs, for instance, by simply re-affixing the absorption portion and the liner portion and changing the insert types, the product can be converted from a pocket diaper into an all-in-two diaper. It is also desired for this convertible reusable diaper to have short dry time and allow the usage of most reusable insert products produced by a wide range of vendors made as well as allowing the usage of biodegradable eco-friendly disposable inserts.

### BRIEF SUMMARY

In an exemplary embodiment, a convertible reusable diaper is put together in a three-part construction, comprised of an outer cover portion, a liner portion, and an absorption portion. The impermeable outer cover portion laminated with either a soft pile or a waterproof breathable material may be operably configured to be positioned on a wearer's body and be releasably affixed thereto, including a frontal region, a crotch region, and a posterior region. A barrier panel portion may be attachable to the outer cover portion. The barrier panel portion generally comprises frontal and posterior barrier panels attachable at the frontal and posterior regions of the outer cover portion to form interior pockets. The interior pockets allow usage of most disposable and reusable insert products produced by a wide range of vendors. One or more first fastener members attachable to the barrier panel portion affix the liner portion or the absorption portion in place. The liner portion comprises a plurality of second fastener members (liner fastener members) cooperatively engaged with the corresponding first fastener members located at the barrier panel portion to allow the liner portion to be removably affixed to the outer cover portion. The liner portion is configured to wick moisture from a wearer's body, and fabricated at least in part from liquid-permeable materials that have a short dry time. Other options for the liner portion comprise natural materials such as cotton, hemp, bamboo, etc. for wearers with exceptionally sensitive skin or for environmentally minded purchasers. The liner portion may be elasticized in any suitable manner to provide a contoured fit around wear's body for preventing leakage. The absorption portion, comprised of at least one insert pad, may be laid onto the outer cover portion or be releasably affixed to the outer cover portion by fastening into the first fastener members located at the barrier panel portion. The insert pads come in two size options: long and regular. The regular insert pads have a plurality of the second fastener members (absorption fastener members) located at one end, which are fastening into the first fastener members located at either the frontal barrier panel or the posterior barrier panel. The long insert pads are designed to allow for quicker dry time. In the open configuration, the long insert pad is about one half the thickness of the regular insert pad but double the length. In the closed configuration, the long insert pad is about the same size and shape as the regular insert pad. The second fastener members (absorption fastener members) on the long insert pad are located at just below the midpoint of the lengthwise sides of the long insert pads. The hole-openings are formed on the barrier panel portion so that the first fastener members can be affixed through the hole-openings. The second fastener members on the liner portion and absorption portion affix to the first fastener members directly or through the hole-openings on either the frontal barrier panel or the posterior barrier panel thereby the liner portion and the absorption portion can be affixed either on the outside or the inside of the interior pockets. Numerous options for affixing the absorption portion and the liner portion allow easy conversion of the diaper from one design to another. The convertible reusable diaper can serve as a waterproof diaper cover and transform into a pocket diaper and an all-in-two diaper via releasably reaffixing the absorption portion and the liner portion.

In another exemplary embodiment, a convertible reusable diaper comprises an outer cover portion, an absorption portion, and a barrier panel portion. The outer cover portion may generally comprise a frontal region, a crotch region, and a posterior region. The absorption portion semi-attached to the outer cover portion may comprise an attachable region securely attached to the inner surface of the outer cover portion, and a detachable region insecurely detached to the inner surface of the outer cover portion. The barrier panel portion comprises a plurality of first fastener members to affix the detachable region of the absorption portion in place. The hole-openings may be formed on the barrier panel portion so that the first fastener members can be affixed through the hole-openings. The detachable region of the absorption portion may further comprise one or more absorption fastener members (second fastener members) cooperatively engaged with the first fastener members to allow the detachable region of the absorption portion to be attachable to the outer cover portion. The barrier panel portion may comprise either a frontal barrier panel or a posterior barrier panel. The frontal barrier panel is attachable to the inner surface of the frontal region of the outer cover portion, and the posterior barrier panel is attachable to the inner surface of the posterior region of the outer cover portion. In one example, the attachable region of the absorption portion is securely attached to the inner surface of the frontal region of the outer cover portion, and the absorption fastener members located at the detachable region of the absorption portion are fastening into the first fastener members located at the inner surface of the posterior barrier panel through the corresponding hole-openings on the posterior barrier panel. In another example, the attachable region of the absorption portion is securely attached to the inner surface of the posterior region of the outer cover portion, and the absorption fastener members located at the detachable region of the absorption portion are fastening into the first fastener members located at the inner surface of the frontal barrier panel through the corresponding hole-openings on the frontal barrier panel. The convertible reusable diaper may further comprise a liner portion securely attached to the absorption portion. The liner portion can be affixed to the outer cover portion along with the detachable region of the absorption portion.

In another exemplary embodiment, a convertible reusable diaper comprises an outer cover portion, a liner portion, and a barrier panel portion. The outer cover portion may generally comprise a frontal region, a crotch region, and a posterior region. The liner portion semi-attached to the outer cover portion may comprise an attachable region securely attached to the inner surface of the outer cover portion, and a detachable region insecurely detached to the inner surface of the outer cover portion. The barrier panel portion comprises a plurality of first fastener members to affix the detachable region of the liner portion in place. The hole-openings may be formed on the barrier panel portion so that the first fastener members can be affixed through the hole-openings. The detachable region of the liner portion may further comprise one or more liner fastener members (second fastener members) cooperatively engaged with the first fastener members to allow the detachable region of the liner portion to be attachable to the outer cover portion. The barrier panel portion may comprise either a frontal barrier panel or a posterior barrier panel. The frontal barrier panel is attached to the inner surface of the frontal region of the outer cover portion, and the posterior barrier panel is attached to the inner surface of the posterior region of the outer cover portion. In one example, the attachable region of the liner portion is securely attached to the inner surface of the frontal region of the outer cover portion, and the liner fastener members located at the attachable region of the liner portion are fastening into the first fastener members located at the inner surface of the posterior barrier panel through the corresponding hole-openings on the posterior barrier panel. In another example, the attachable region of the liner portion is securely attached to the inner surface of the posterior region of the outer cover portion, and the liner fastener members located at the detachable region of the liner portion are fastening into the first fastener members located at the inner surface of the frontal barrier panel through the corresponding hole-openings on the frontal barrier panel. The convertible reusable diaper may further comprise an absorption portion including at least one insert pad that comes in two size options: standard and extended. The standard insert pad is securely attached to the liner portion and can be affixed to the outer cover portion along with the detachable region of the line portion. The extended insert pad comprises a base region securely attached to the liner portion, and an extended region openly detached to the liner portion. In the close configuration, the extended region of the long insert pad folds into the inner side of the outer cover portion. The extended insert pad is designed to allow for more absorbency and quicker dry time.

Further aspects and features of the present invention will be explained in details in the detailed description section of the document. It should be understood that the detailed description and specific examples indicating exemplary embodiment of the present invention are intended for purposes of illustration only and are not intended to limit the scope of the present invention.

### BRIEF DESCRIPTION OF THE DRAWINGS

The drawings described herein are for illustration purpose only and are not intended to limit the scope of the present disclosure in any way.

FIG. 1 is a perspective view of an outer cover portion of a convertible reusable diaper with a liner portion and an absorption portion installed according to exemplary embodiments of the disclosure;

FIG. 2 is a top view of an outer cover portion of a convertible reusable diaper with a liner portion and an absorption portion installed according to exemplary embodiments of the disclosure;

FIG. 3 is a top view of an outer cover portion of a convertible reusable diaper with a frontal barrier panel and a posterior barrier panel according to exemplary embodiments of the disclosure;

FIG. 4 is top view of an outer cover portion of a convertible reusable diaper with a regular insert pad installed on a posterior barrier panel of the outer cover portion according to exemplary embodiments of the disclosure;

FIG. 5 illustrates a first fastener member and its corresponding hole-opening according to exemplary embodiments of the disclosure;

FIG. 6 is a top view of a liner portion of a convertible reusable diaper according to exemplary embodiments of the disclosure;

FIG. 7 is a top view of an outside of an outer cover portion of a convertible reusable diaper according to exemplary embodiments of the disclosure;

FIG. 8 is a top view of a regular insert pad of a convertible reusable diaper according to exemplary embodiments of the disclosure;

FIG. 9 is a top view of a long insert pad of a convertible reusable diaper according to exemplary embodiments of the disclosure, shown in an open configuration;

FIG. 10 is a top view of a long insert pad of a convertible reusable diaper according to exemplary embodiments of the disclosure, shown in a closed configuration;

FIG. 11 is a perspective view of an outer cover portion of a convertible reusable diaper with a liner portion and an absorption portion semi-attached to the outer cover portion according to exemplary embodiments of the disclosure;

FIG. 12 is a top view of an outer side of a liner portion with an extended insert pad according to exemplary embodiments of the disclosure;

FIG. 13 is a top view of an inner side of a liner portion with an extended insert pad according to exemplary embodiments of the disclosure;

FIG. 14 is a top view of an outer cover portion of a convertible reusable diaper with a frontal barrier panel according to exemplary embodiments of the disclosure; and

FIG. 15 is a top view of an outer cover portion of a convertible reusable diaper with a liner portion and an absorption portion semi-attached to the outer cover portion according to exemplary embodiments of the disclosure, shown in a closed configuration.

### DETAILED DESCRIPTION OF THE INVENTION

While this inventions is susceptible of embodiment in many different forms, there is shown in the drawings, and described in detail herein, one embodiment, with the understanding that the present disclosure is to be considered an exemplification of the principles of the invention and is not intended to limit the invention to the embodiment illustrated.

The following description and drawings are illustrative and are not to be construed as limiting. Numerous specific details are described to provide a thorough understanding of the disclosure. However, in certain instances, well-known or conventional details are not described in order to avoid obscuring the descriptions. References to one or an embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this disclosure pertains. In the case of conflict, the present document, including definitions will control.

According to various aspects, exemplary embodiments are provided of convertible reusable diapers. In one exemplary embodiment, a convertible reusable diaper may comprise an outer cover portion, and a barrier panel portion. The outer cover portion may generally comprise a frontal region, a crotch region, and a posterior region. The barrier panel portion comprise at least one barrier panel attachable to the outer cover portion. One or more liquid-absorbent reusable or disposal insert pads may be removably disposed between the outer cover portion and the barrier panel portion. The convertible reusable diaper may further comprises one or more first fastener members attachable to the barrier panel portion, and one or more hole-openings formed on the barrier panel portion, therefore the first fastener members can affix the reusable liners and insert pads in place through the hole-openings.

In another exemplary embodiment, a convertible reusable diaper is put together in a three-part construction, comprised of an outer cover portion, a liner portion, and an absorption portion. The impermeable outer cover portion laminated with either a soft pile or a waterproof breathable material may be operably configured to be positioned on a wearer's body and be releasably affixed thereto, including a frontal region, a crotch region, and a posterior region. A barrier panel portion may be attachable to the outer cover portion. The barrier panel portion generally comprises frontal and posterior barrier panels attachable at the frontal and posterior regions of the outer cover portion to form interior pockets. The interior pockets allow usage of most disposable and reusable insert products produced by a wide range of vendors. One or more first fastener members attachable to the barrier panel portion affix the liner portion or the absorption portion in place. The liner portion comprises a plurality of second fastener members (liner fastener members) cooperatively engaged with the corresponding first fastener members located at the barrier panel portion to allow the liner portion to be removably affixed to the outer cover portion. The liner portion is configured to wick moisture from a wearer's body, and fabricated at least in part from liquid-permeable materials that have a short dry time. Other options for the liner portion comprise natural materials such as cotton, hemp, bamboo, etc. for wearers with exceptionally sensitive skin or for environmentally minded purchasers. The liner portion may be elasticized in any suitable manner to provide a contoured fit around wear's body for preventing leakage. The absorption portion, comprised of at least one insert pad, may be laid onto the outer cover portion or be releasably affixed to the outer cover portion by fastening into the first fastener members located at the barrier panel portion. The insert pads come in two size options: long and regular. The regular insert pads have a plurality of the second fastener members (absorption fastener members) located at one end, which are fastening into the first fastener members located at either the frontal barrier panel or the posterior barrier panel. The long insert pads are designed to allow for quicker dry time. In the open configuration, the long insert pad is about one half the thickness of the regular insert pad but double the length. In the closed configuration, the long insert pad is about the same size and shape as the regular insert pad. The second fastener members (absorption fastener members) on the long insert pad are located at just below the midpoint of the lengthwise sides of the long insert pads. The hole-openings are formed on the barrier panel portion so that the first fastener members can be affixed through the hole-openings. The second fastener members on the liner portion and absorption portion affix to the first fastener members directly or through the hole-openings on either the frontal barrier panel or the posterior barrier panel thereby the liner portion and the absorption portion can be affixed either on the outside or the inside of the interior pockets. Numerous options for affixing the absorption portion and the liner portion allow easy conversion of the diaper from one design to another. The convertible reusable diaper can serve as a waterproof diaper cover and transform into a pocket diaper and an all-in-two diaper via releasably reaffixing the absorption portion and the liner portion.

In another exemplary embodiment, a convertible reusable diaper comprises an outer cover portion and an absorption portion. The outer cover portion may generally comprise a frontal region, a crotch region and a posterior region. The outer cover portion may further comprise a barrier panel portion that may comprise a frontal barrier panel and/or a posterior barrier panel. The frontal barrier panel is attached to the inner surface of the frontal region of the outer cover portion, and the posterior barrier panel is attached to the inner surface of the posterior region of the outer cover. One or more first fastener members can be attached to either the inner surface of the outer cover portion or the frontal and/or posterior barrier panels when the frontal and/or posterior barrier panels are included in the outer cover portion. The absorption portion comprises one or more absorption fastener members (second fastener members). At least one of the absorption fastener members is cooperatively engaged with one of the first fastener members to allow the absorption portion to be removably affixed to the outer cover portion. The hole-openings may be formed on the frontal and/or posterior barrier panels allowing the absorption fastener members to be fastening into the first fastener members located at either the inner surface of the outer cover portion, or the frontal and/or posterior barrier panels through the hole-openings. The absorption portion is configured to be saturated with a diaper wearer's bodily discharges, and fabricated, at least in part, from a liquid-absorbent material. The absorption portion may further comprise an outer layer which is configured to wick moisture from a diaper wearer's body, and fabricated at least in part from a liquid-permeable material.

In another exemplary embodiment, a convertible reusable diaper comprises an outer cover portion and a liner portion. The outer cover portion may generally comprise a frontal region, a crotch region, a posterior region. The outer cover portion may further comprise a barrier panel portion that may comprise a frontal barrier panel and/or a posterior barrier panel. The frontal barrier panel is attached to the inner surface of the frontal region of the outer cover portion, and the posterior barrier panel is attached to the inner surface of the posterior region of the outer cover. One or more first fastener members can be attached to either the inner surface of the outer cover portion or the frontal and/or posterior barrier panels when the frontal and/or posterior barrier panels are included in the outer cover portion. The liner portion comprises one or more liner fastener members (second fastener members). At least one of the liner fastener members is cooperatively engaged with one of the first fastener members to allow the liner portion to be removably affixed to the outer cover portion. The hole-openings may be formed on the frontal and/or posterior barrier panels allowing the liner fastener members to be fastening into the first fastener members located at either the inner surface of the outer cover portion, or the frontal and/or posterior barrier panels through the hole-openings. The liner portion is fabricated, at least in part, from at least one of: a liquid-permeable material configured to wick moisture from a diaper wearer's body, and a natural material configured to reduce irritation of skin of a diaper wearer's body. The liner portion may be elasticized in any suitable manner to provide a contoured fit around a wearer's body for preventing leakage. The convertible reusable diaper may further comprise at least one removable insert pad which is fabricated from materials that are reusable, washable, disposable, and/or biodegradable. The removable insert pad may be positioned between the outer cover portion and the liner portion, or deposed within the liner portion. There may be provided a plurality of replacement liners in the liner portion or the removable insert pads. In such embodiments, an existing liner or the removable insert pad may be removed from the outer cover portion after the removable insert pad has become saturated. One of the replacement liners and the removable insert pads can be positioned within the outer cover portion after the diaper has been washed or laundered.

In another exemplary embodiment, the convertible reusable diaper comprises an outer cover portion and a liner portion. The outer cover portion may generally comprise a frontal region, a crotch region, and a posterior region. The liner portion semi-attached to the outer cover portion may comprise an attachable region securely attached to the inner surface of the outer cover portion, and a detachable region insecurely detached to the inner surface of the outer cover portion. A plurality of first fastener members may be placed on the inner surface of the outer cover portion. The detachable region of the liner portion may further comprise one or more liner fastener members (second fastener members) cooperatively engaged with the first fastener members to allow the detachable region of the liner portion to be attachable to the outer cover portion. In one example, the attachable region of the liner portion is securely attached to the inner surface of the frontal region of the outer cover portion, and the first fastener members may be placed on the inner surface of the posterior and/or crotch regions of the outer cover portion. In another example, the attachable region of the liner portion is securely attached to the inner surface of the posterior region of the outer cover portion, and the first fastener members are placed on the inner surface of the frontal and/or crotch regions of the outer cover portion. The convertible reusable diaper may further comprise an absorption portion comprised of at least one insert pad. The insert pad comes in two size options: standard and extended. The standard insert pad is securely attached to the liner portion and can be attachable to the outer cover portion along with the detachable region of the line portion. The extended insert pad comprises a base region securely attached to the liner portion, and an extended region openly detached to the liner portion and the outer cover portion. In the close configuration, the extended region of the extended insert pad folds into the inner side of the outer cover portion. The extended insert pad is designed to allow for more absorbency and quicker dry time.

In another exemplary embodiment, the convertible reusable diaper comprises an outer cover portion and an absorption portion. The outer cover portion may generally comprise a frontal region, a crotch region, and a posterior region. The absorption portion semi-attached to the outer cover portion may comprise an attachable region securely attached to the inner surface of the outer cover portion, and a detachable region insecurely detached to the inner surface of the outer cover portion. A plurality of first fastener members may be placed on the inner surface of the outer cover portion. The detachable region of the absorption portion may further comprise one or more absorption fastener members (second fastener members) cooperatively engaged with the first fastener members to allow the detachable region of the absorption portion to be attachable to the outer cover portion. In one example, the attachable region of the absorption portion is securely attached to the inner surface of the frontal region of the outer cover portion, and the first fastener members may be placed on the inner surface of the posterior and/or crotch regions of the outer cover portion. In another example, the attachable region of the absorption portion is securely attached to the inner surface of the posterior region of the outer cover portion, and the first fastener members are placed on the inner surface of the frontal and/or crotch regions of the outer cover portion. The convertible reusable diaper may further comprise a liner portion securely attached to the absorption portion. The liner portion may be placed between a wearer's body and the absorption portion to provide a stay-dry layer to wick moisture from the wearer's body, or a gentle, non-irritating layer for the sensitive skin.

In another exemplary embodiment, a convertible reusable diaper comprises an outer cover portion, an absorption portion, and a barrier panel portion. The outer cover portion may generally comprise a frontal region, a crotch region, and a posterior region. The absorption portion semi-attached to the outer cover portion may comprise an attachable region securely attached to the inner surface of the outer cover portion, and a detachable region insecurely detached to the inner surface of the outer cover portion. The barrier panel portion comprises a plurality of first fastener members to affix the detachable region of absorption portion in place. The hole-openings may be formed on the barrier panel portion so that the first fastener members can be affixed through the hole-openings. The detachable region of the absorption portion may further comprise one or more absorption fastener members (second fastener members) cooperatively engaged with the first fastener members to allow the detachable region of the absorption portion to be attachable to the outer cover portion. The barrier panel portion may comprise either a frontal barrier panel or a posterior barrier panel. The frontal barrier panel is attachable to the inner surface of the frontal region of the outer cover portion, and the posterior barrier panel is attachable to the inner surface of the posterior region of the outer cover portion. In one example, the attachable region of the absorption portion is securely attached to the inner surface of the frontal region of the outer cover portion, and the absorption fastener members located at the detachable region of the absorption portion are fastening into the first fastener members located at the inner surface of the posterior barrier panel through the corresponding hole-openings on the posterior barrier panel. In another example, the attachable region of the absorption portion is securely attached to the inner surface of the posterior region of the outer cover portion, and the absorption fastener members located at the detachable region of the absorption portion are fastening into the first fastener members located at the inner surface of the frontal barrier panel through the corresponding hole-openings on the frontal barrier panel. The convertible reusable diaper may further comprise a liner portion securely attached to the absorption portion. The liner portion can be affixed to the outer cover portion along with the detachable region of the absorption portion.

In another exemplary embodiment, a convertible reusable diaper comprises an outer cover portion, a liner portion, and a barrier panel portion. The outer cover portion may generally comprise a frontal region, a crotch region, and a posterior region. The liner portion semi-attached to the outer cover portion may comprise an attachable region securely attached to the inner surface of the outer cover portion, and a detachable region insecurely detached to the inner surface of the outer cover portion. The barrier panel portion comprises a plurality of first fastener members to affix the detachable region of liner portion in place. The hole-openings may be formed on the barrier panel portion so that the first fastener members can be affixed through the hole-openings. The detachable region of the liner portion may further comprise one or more liner fastener members (second fastener members) cooperatively engaged with the first fastener members to allow the detachable region of the liner portion to be attachable to the outer cover portion. The barrier panel portion may comprise either a frontal barrier panel or a posterior barrier panel. The frontal barrier panel is attached to the inner surface of the frontal region of the outer cover portion, and the posterior barrier panel is attached to the inner surface of the posterior region of the outer cover portion. In one example, the attachable region of the liner portion is securely attached to the inner surface of the frontal region of the outer cover portion, and the liner fastener members located at the attachable region of the liner portion are fastening into the first fastener members located at the inner surface of the posterior barrier panel through the corresponding hole-openings on the posterior barrier panel. In another example, the attachable region of the liner portion is securely attached to the inner surface of the posterior region of the outer cover portion, and the liner fastener members located at the detachable region of the liner portion are fastening into the first fastener members located at the inner surface of the frontal barrier panel through the corresponding hole-openings on the frontal barrier panel. The convertible reusable diaper may further comprise an absorption portion including at least one insert pad that comes in two size options: standard and extended. The standard insert pad is securely attached to the liner portion and can be affixed to the outer cover portion along with the detachable region of the line portion. The extended insert pad comprises a base region securely attached to the liner portion, and an extended region openly detached to the liner portion. In the close configuration, the extended region of the long insert pad folds into the inner side of the outer cover portion. The extended insert pad is designed to allow for more absorbency and quicker dry time.

In some exemplary embodiments, the outer cover portion, the liner portion, the absorption portion, and the barrier panel portion are configured to be reusable, and fabricated from washable materials. The outer cover portion may further comprise an outer shell which is preferably fabricated from a breathable water proof or water resistant material such as polyester, fleece, wool, and thermoplastic polyurethane laminated material. The liner portion may be fabricated from either natural materials, such as linen, cotton, jute, hemp, bamboo, kenaf, sisal, combinations thereof, etc., or liquid-permeable materials, such as polar fleece, fleece, micro fleece, suede cloth, combinations thereof, etc. The barrier panel portion including the frontal and posterior barrier panels may be fabricated from the same material as the one used by either the outer shell or the liner portion, such as polyester, fleece, wool, thermoplastic polyurethane laminated material, polar fleece, fleece, micro fleece, suede cloth, linen, cotton, jute, hemp, bamboo, kenaf and sisal. The absorption portion may be fabricated from a liquid absorbent material, such as linen, hemp, cotton, microfiber, bamboo, polyester or any artificial and synthetic fibre fabric. The fastener members including the first and second fastener members (liner fastener members and absorption fastener members) may be selected from a group comprising of adhesives, buttons, clasps, hook and loop closures, magnets, elastic straps, adjustable straps, combinations thereof, etc.

In some exemplary embodiments, the outer cover portion may generally comprise a frontal region, a crotch region and a posterior region. The posterior region comprises posterior adjustable fastener members located at top corner areas, and the frontal region comprises frontal adjustable fastener members for being releasably affixed to the posterior region via cooperatively engaging into the corresponding posterior adjustable fastener members in the posterior region to position the convertible diaper on a diaper wearer's body and allowing adjustable waist size of the convertible reusable diaper. Still further embodiments may comprise a wide range of other suitable fastening systems besides snaps, such as adhesives, buttons, clasps, hook and loop closures, magnets, elastic straps, adjustable straps, combinations thereof, etc.

In some exemplary embodiments, the outer cover portion may generally comprise a frontal region, a crotch region and a posterior region. The frontal region comprises a plurality of adjustable fastener members cooperatively engageable to allow adjustable size options on the convertible reusable diaper. In one example, at least a two-by-three array of adjustable fastener members along the frontal region that allows selective adjustment to a functional rise of the convertible reusable diaper. The array comprises at least of first row of at least two spaced-apart adjustable fastener members vertically spaced from and aligned with corresponding adjustable fastener members in at least two other rows of the array. Still further embodiments may comprise a wide range of other suitable fastening systems besides snaps, such as adhesives, buttons, clasps, hook and loop closures, magnets, elastic straps, adjustable straps, combinations thereof, etc.

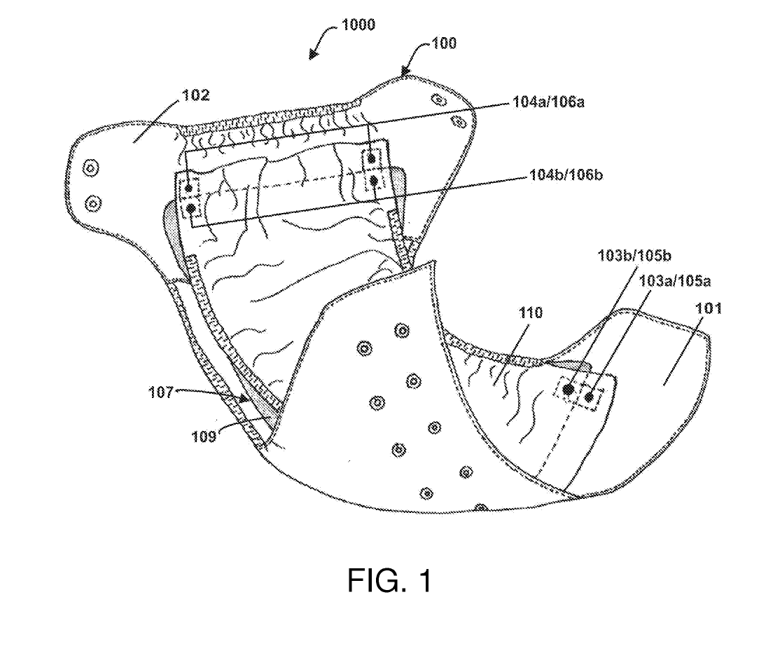

FIGS. 1-15 illustrate convertible reusable diaper constructions, in accordance with exemplary embodiments of the disclosure. Referring now to FIG.1, a convertible reusable diaper 1000 may comprise an outer cover portion 100 and a liner portion 110. The outer cover portion 100 may comprise a frontal barrier panel 101 and a posterior barrier panel 102 which are attached to an inner surface of front and rear top areas of the outer cover portion 100 respectively. The liner portion 110 is releasably affixed to the outer cover portion 100 at four points via fastening through hole-openings 105a/106a into first fastener members 103a/104a located at the frontal barrier panel 101 and posterior barrier panel 102. An absorption portion 107 comprising at least one insert pad 109 may be placed between the liner portion 110 and the outer cover portion 100. The insert pad 109 is releasably affixed to the outer cover portion 100 via fastening through hole-openings 105b/106b into first fastener members 103b/104b located at the frontal panel 101 and the posterior barrier panel 102.

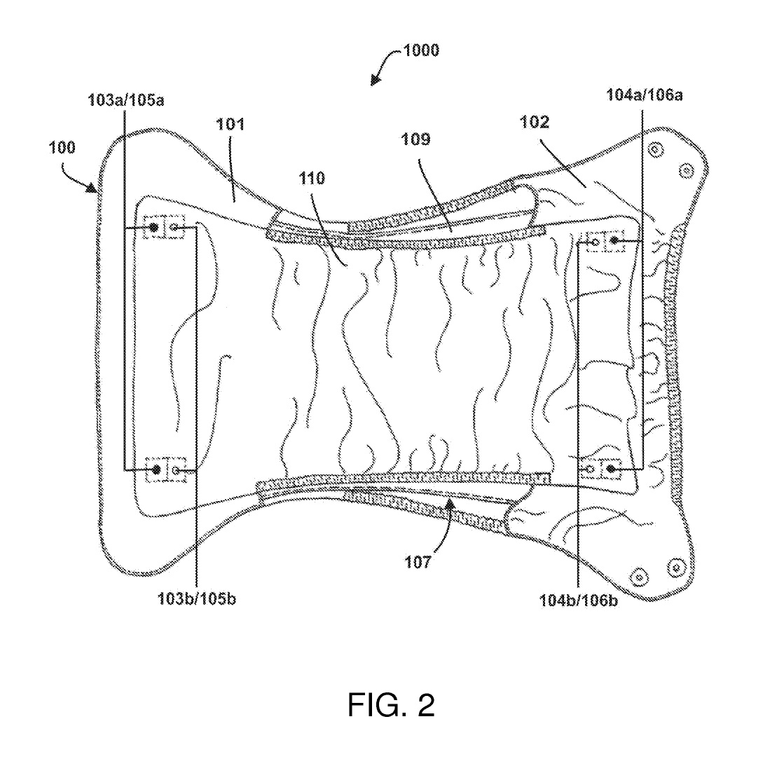

FIG. 2 illustrates a top view of an outer cover portion of a convertible reusable diaper with a liner portion and an absorption portion installed according to exemplary embodiments of the disclosure.

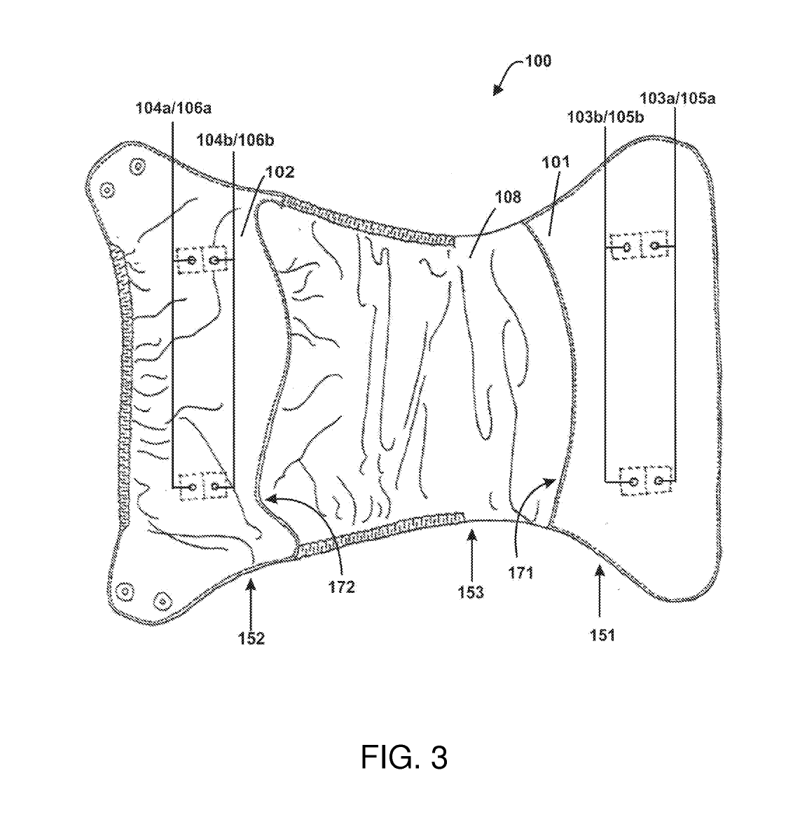

FIG. 3 illustrates a top view of an outer cover portion of a convertible reusable diaper with a frontal barrier panel and a posterior barrier panel according to exemplary embodiments of the disclosure. The outer cover portion 100 may comprise an outer shell 108, which is preferably fabricated from a breathable water proof or water resistant material. The frontal barrier panel 101 and the posterior barrier panel 102 may be preferably fabricated from natural materials (such as linen, cotton, jute, hemp, bamboo, kenaf, sisal, etc.), which are configured to minimize irritation of skin of a diaper wearer's body. The frontal barrier panel 101 and the posterior barrier panel 102 may also be fabricated from liquid-permeable materials (such as polar fleece, fleece, micro fleece, suede cloth, etc.), which have a short dry time and are configured to wick moisture from a diaper wearer's body. Moreover, the frontal barrier panel 101 and the posterior barrier panel 102 may be fabricated from a breathable water proof or water resistant material for preventing leakage. The frontal barrier panel 101 may be attached to an inner surface of a frontal region 151 to form a frontal pocket 171. The posterior barrier panel 102 may be attached to an inner surface of a posterior region 152 to form a posterior pocket 172. One or more reusable or disposable absorbent pads may be disposed within the outer cover portion 100 with two opposite edges placed and retained inside of the frontal pocket 171 and the posterior pocket 172. The first fastener members 103a/104a and 103b/104b are attached underneath to the hole-openings 105a/106a and 105b/106b located on the frontal barrier panel 101 and the posterior barrier panel 102. The first fastener members 103a/104a and 103b/104b may be placed inside of the frontal pocket 171 and the posterior pocket 172 to prevent the first fastener members 103a/104a and 103b/104b from being exposed to a diaper wearer's skin when the liner portion 110 or the absorption portion 107 (shown in FIG. 1)is not affixed with the outer cover 100. Therefore, the outer cover 100 can be used as a reusable diaper shell.

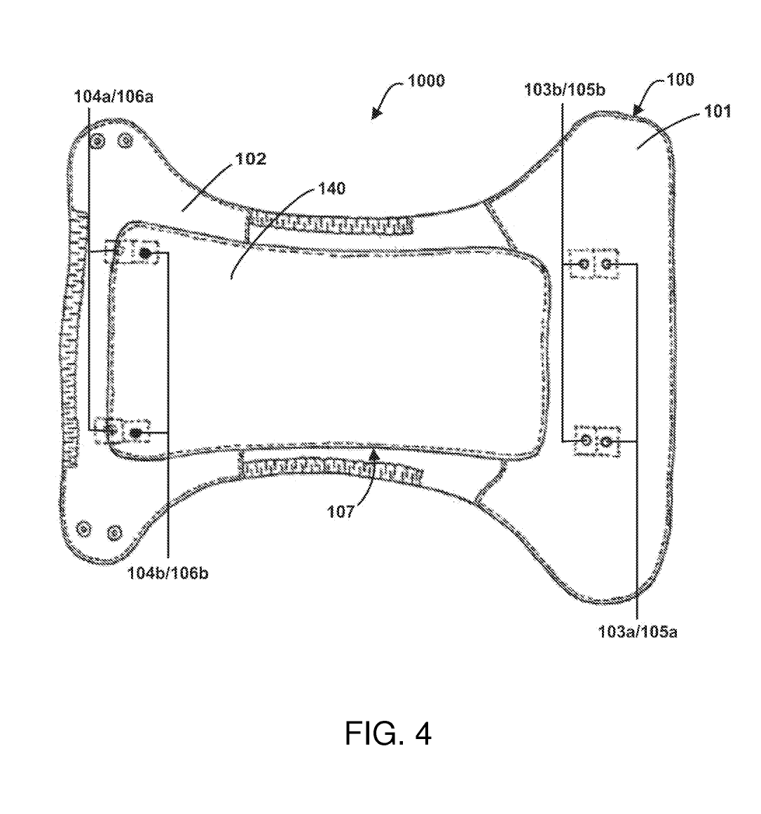

FIG. 4 is top view of an outer cover portion of a convertible reusable diaper with a regular insert pad installed on a posterior barrier panel of the outer cover portion according to exemplary embodiments of the disclosure. As an embodiment shown in FIG. 4, the absorption portion 107 may comprise at least one regular insert pad 140. The regular pad insert 140 may have one edge releasably affixed to either the frontal barrier panel 101 or the posterior barrier panels 102 of the outer cover portion 100. An opposite edge and two sides of the regular insert pad 140 are not fastened to the frontal barrier panel 101 and posterior barrier panel 102 or the outer cover portion 100. As shown in FIG. 4, the regular insert pad of 140 has one edge releasably affixed to the outer cover portion by fastening into the first fastener members 104b through the hole-openings 106b located at the posterior barrier panel 102. Using the same method, another regular insert pad may be releasably affixed to the frontal barrier panel 101. Therefore, the regular insert pad 140 is stacked on top of another regular insert pad to allow customized absorbency.

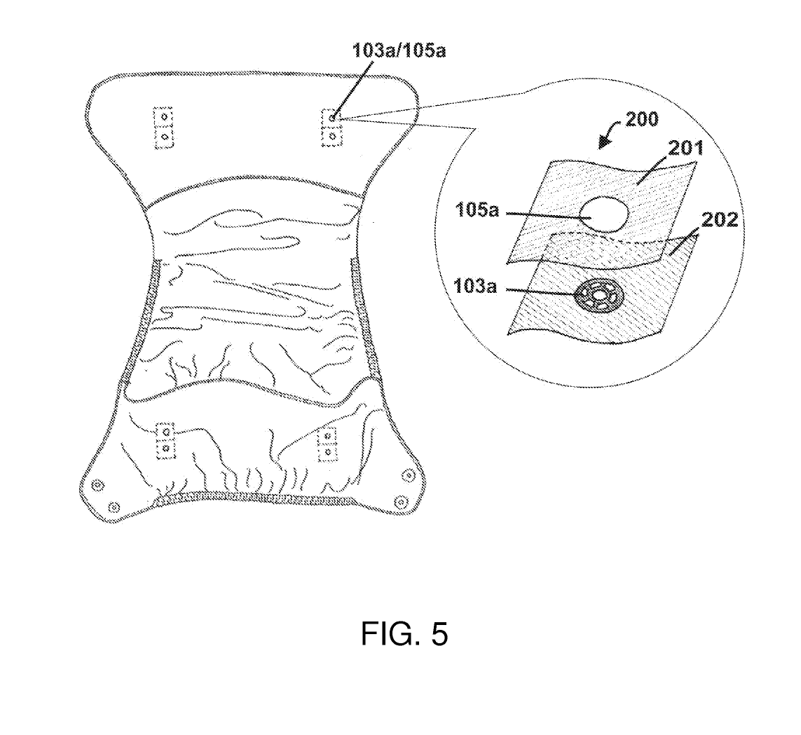

FIG. 5 illustrates a first fastener member and its corresponding hole-opening according to exemplary embodiments of the disclosure. As shown in FIG. 5, an enlarge illustration 200 demonstrates how the hole-opening and the first fastener members work together to provide fastenable unit on the frontal barrier panel. The hole-opening 105a is located on the frontal barrier panel. A fabric 201 around the hole-opening 105a is part of the frontal barrier panel. The first fastener member 103a is affixed on a tab 202, and the tab 202 is attached underneath the hole-opening 105a. The hole-opening 105a provides a hole or opening on top of the first fastener member 103a through the frontal barrier panel so that the first fastener member 103a may be reached and affixed through the hole-opening 105a.

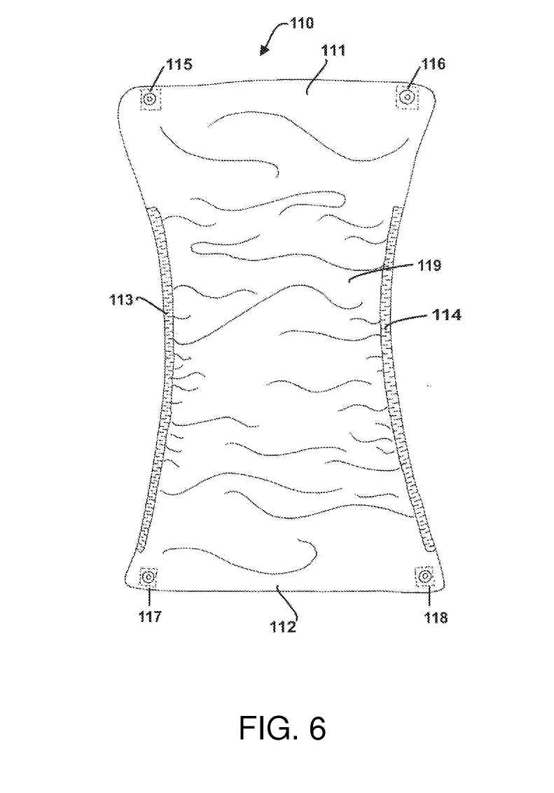

In another embodiment as shown in FIG. 6, the liner portion 110 may provide a layer of fabric, liner 119, between a diaper wearer's body and liquid-absorbent insert pads. The liner 119 may be fabricated from a natural material (such as linen, cotton, jute, hemp, bamboo, kenaf, sisal, etc.) or a liquid-permeable material (such as polar fleece, fleece, micro fleece, suede cloth, etc.). The liner portion 110 may generally comprise a frontal edge 111, a posterior edge 112, and elastic disposed adjacent side edges 113 and 114. Second fastener members comprise a plurality of liner fastener members 115, 116, 117, 118 attached to four corners of the liner portion 110. The liner fastener members 115, 116 located at the frontal edge 111 can be cooperatively engaged with the first fastener members 103a/103b located at the frontal barrier panel 101. The liner fastener members located at the posterior edge 112 may be cooperatively engaged with the first fastener members 104a/104b located at the posterior barrier panel 102. Alternatively, the liquid-absorbent insert pads can be deposed within the liner portion 110.

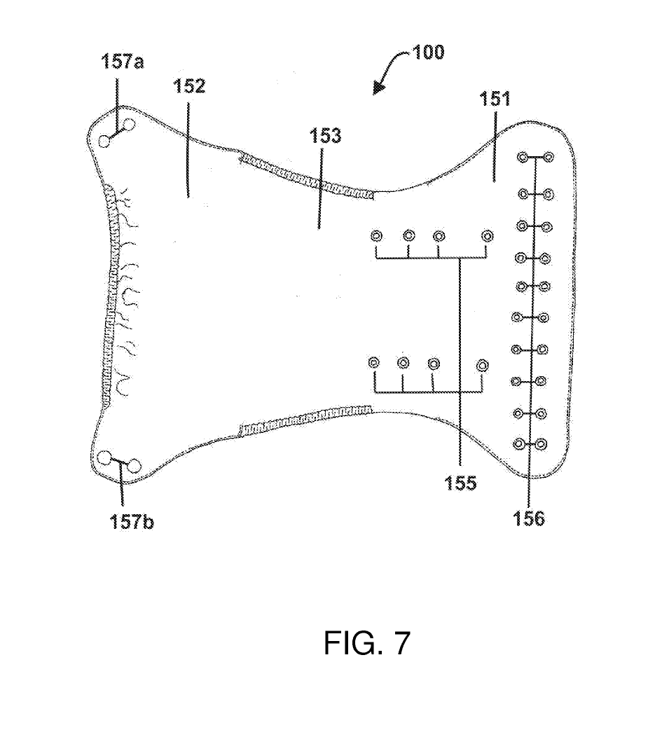

FIG. 7 is a top view of the outside of the outer cover portion 100. The outer cover portion 100 comprises the frontal region 151 and the posterior region 152. The frontal region 151 comprises a plurality of adjustable fastener members 155 cooperatively engageable to allow adjustable size options on the convertible reusable diaper 1000. As shown in FIG. 7, at least one two-by-three array of adjustable fastener members 155 along the frontal region 151 comprises at least a first row of at least one space-apart adjustable fastener member 155 vertically spaced from and aligned with the corresponding adjustable members 115 in at least one other rows of the array. The posterior region 152 comprises a plurality of posterior adjustable fastener members 157a, 157b located at top corner areas of the outer cover portion 100. The frontal region 151 further comprises a plurality of frontal adjustable fastener members 156 for being releasably affixed to the posterior region 152 via engaging into the corresponding posterior adjustable fastener member 157a, 157b in the corner areas of the posterior region 152 to position the convertible reusable diaper 1000 on a diaper wearer's body and allow adjustable waist size of the convertible reusable diaper 1000. In some preferred embodiments, the frontal region 151 may be releasably attached to the posterior region 152 by using hook-and-loop fasteners. Additional embodiments, however, may alternatively comprise snaps, pins, buttons, adhesives, clasps, magnets, combinations thereof, etc. The outer cover portion 100 further includes a crotch region 153 comprising two elastic disposed adjacent side edges.

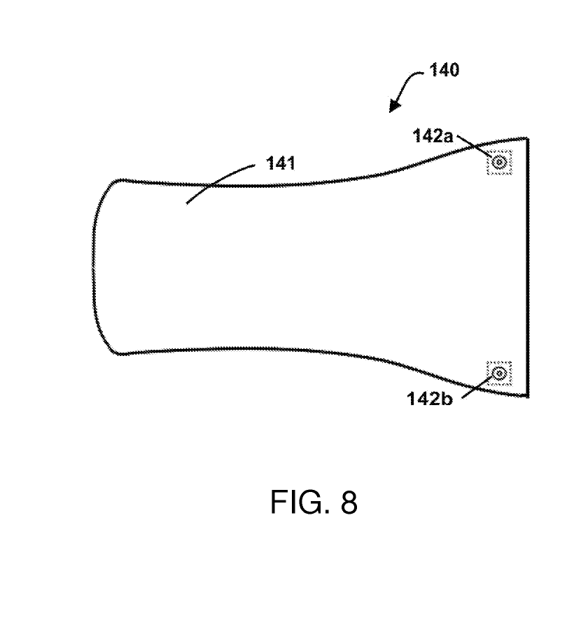

FIG. 8 is a top view of the regular insert pad 140 of a convertible reusable diaper according to exemplary embodiments of the disclosure. In FIG. 8, the regular insert pad 140 may comprise a plurality of absorption fastener members 142a, 142b located at corners of one edge. The absorption fastener members 142a, 142b can be cooperatively engaged with either the first fastener members located at the frontal barrier panel or the first fastener members located at the posterior barrier panel. The regular insert pad 140 may further comprise an outer layer 141 which is configured to wick moisture from a diaper wearer's body, and is fabricated at least in part from a liquid-permeable material, such as polar fleece, fleece, micro fleece, suede cloth, etc.

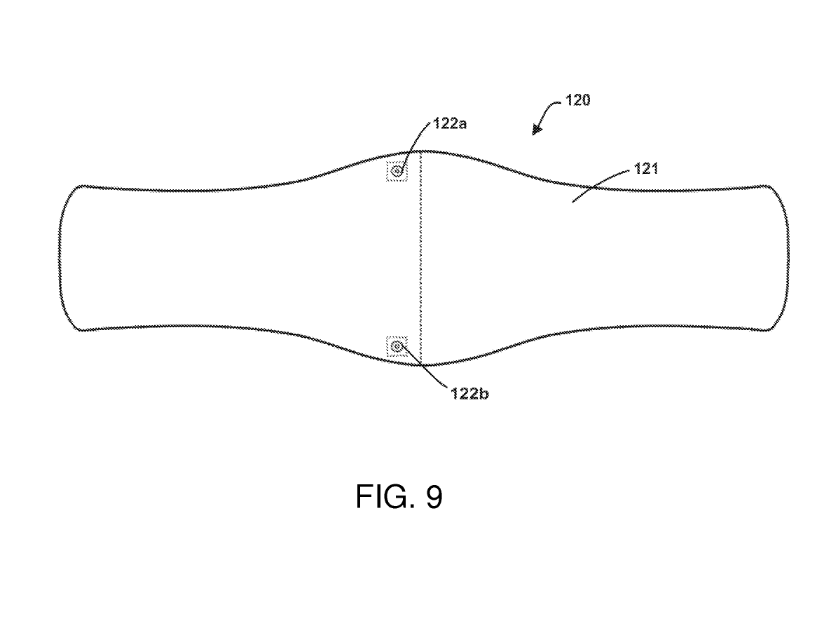

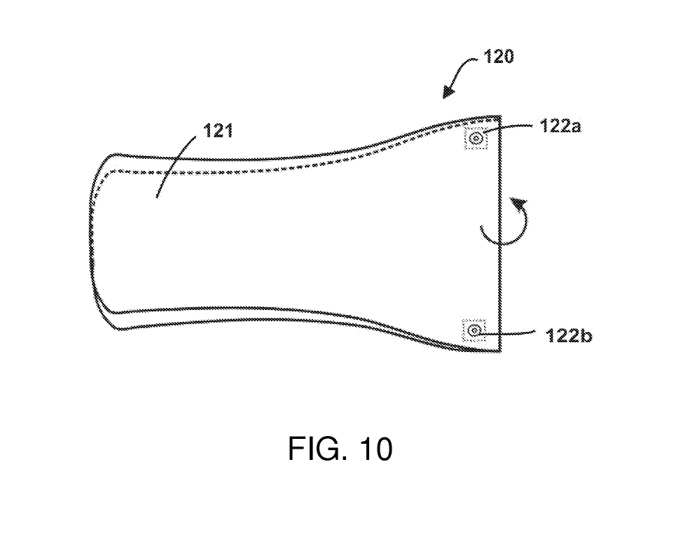

The regular insert pad 140 shown in FIG. 8 may be used in combination with a long insert pad in one embodiment or may be replaced by a long insert pad in another embodiment. Depending on various applications, the insert pad 109 (shown in FIGS. 1 and 2), the long insert pad 120 and the regular insert pad 140 may be used in one exemplary embodiment. In other embodiments, each of them may be used independently. In other words, the absorption portion 107 (shown in FIG. 1) may comprise at least one of the insert pad 109, the long insert pad 120 and the regular insert pad 140. FIG. 9 illustrates a top view of a long insert pad 120 of a convertible reusable diaper according to exemplary embodiments of the disclosure. In FIG. 9, the long insert pad 120 may have a plurality of absorption fastener members 122a, 122b located at just below the midpoint of the lengthwise sides of the long insert pad 120. In the open configuration shown in FIG. 9, the long insert pad 120 may be half the thickness of the regular insert pad but double the length. In the closed configuration shown in FIG. 10, the long insert pad 120 is about the same size and shape as the regular insert pad. The absorption fastener members 122a, 122b can be cooperatively engaged with either the first fastener members located at the frontal barrier panel or the first fastener members located at the posterior barrier panel. The long insert pad 120 may further comprise an outer layer 121 which is configured to wick moisture from a diaper wearer's body, and is fabricated at least in part from a liquid-permeable material, such as polar fleece, fleece, micro fleece, suede cloth, etc.

In some embodiments, one or two regular insert pads 140 can be removably affixed to the outer cover portion 100. It builds up the convertible reusable diaper 1000 with attached reusable washable liquid-absorbent insert(s).

In some embodiments, one or two long insert pads 120 may be removably affixed to the outer cover portion 100. It builds up a convertible reusable diaper 1000 with attached reusable washable liquid-absorbent insert(s).

And in other embodiments, the regular insert pad and the long insert pad 120 may be mixed together to be removably affixed to the outer cover portion 100 to build another reusable washable liquid-absorbent cloth diaper.

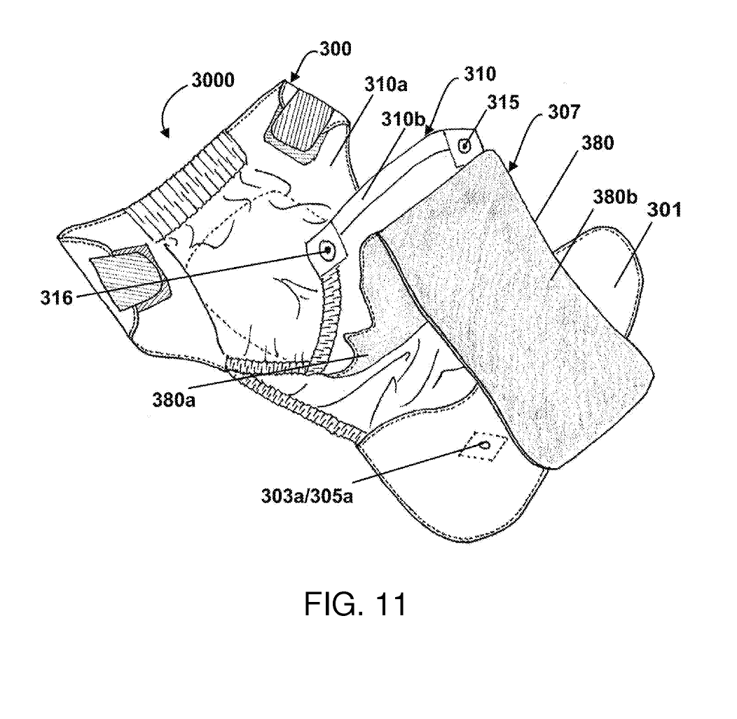

As an embodiment shown in FIG. 11, a convertible reusable diaper 3000, in embodiments of the invention, may comprise an outer cover portion 300, a liner portion 310 semi-attached to the outer cover portion 300, and an absorption portion 307 comprising one extended insert pad 380. The outer cover portion 300 may comprise a frontal barrier panel 301 attached to an inner surface of the front top areas of the outer cover portion 300. An attachable region 310a of the liner portion 310 may be affixed to an inner surface of the posterior top areas of the outer cover portion 300. A detachable region 310b of the liner portion 310 may comprise a plurality of liner fastener members 315/316 at top corner areas. A base region 380a of the extended insert pad 380 may be affixed to the liner portion 310, and an extended region 380b of the extended insert pad 380 may not be fastened or affixed to the outer cover portion 300 or the liner portion 310. The detachable region 310b of the liner portion 310 may be detachably affixed to the outer cover portion 300 via fastening through hole-openings 305a into first fastener members 303a located at the frontal barrier panel 301, covering the extended insert pad 380 with the extended region 380b placed and retained between the liner portion 310 and the outer cover portion 300.

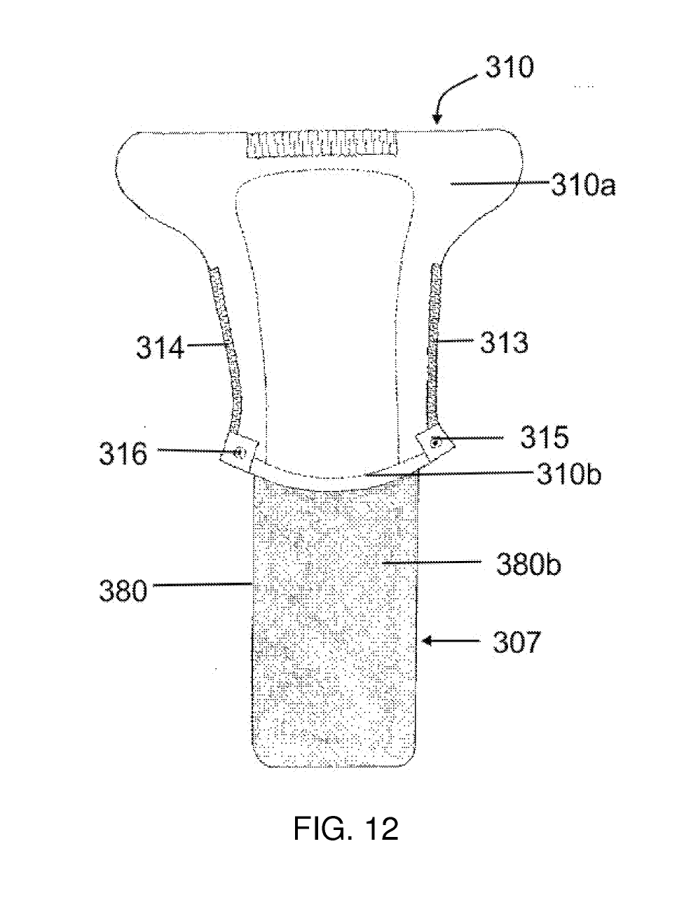

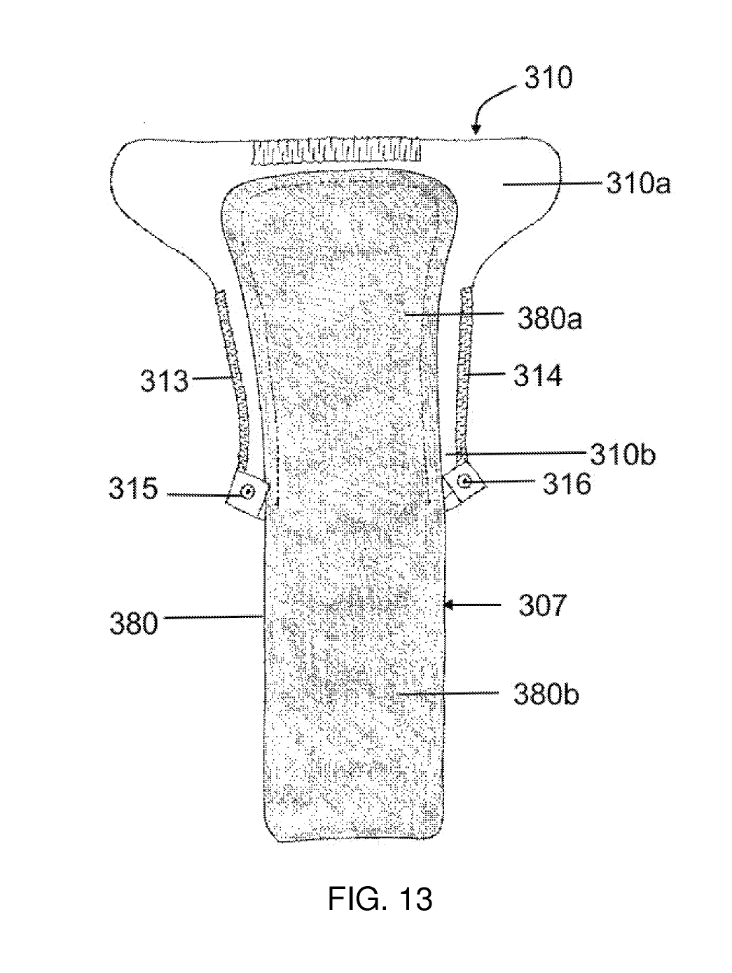

FIG. 12 is a top view of an outside of the liner portion 310 with the extended insert pad 380 of the absorption portion 307. FIG. 13 is a top view of an inside of the liner portion 310 with the extended insert pad 380 of the absorption portion 307. With references to FIG. 12 and FIG. 13, the detachable region 310b of the liner portion 310 comprises a plurality of liner fastener members 315/316 at top corner areas. The base region 380a of the extended insert pad 380 has one edge affixed to the attachable region 310a of the liner portion 310. Two sides of the base region 310a of the extended insert pad 380 are affixed to the areas between elastic side edges 313 and 314 of the liner portion 310. The rest part of the base region 310a of the extended insert pad 380 may be either attached or detached to the liner portion 310. The extended region 380b of the extended insert pad 380 is extended from the base region 380a of the extended insert pad 380 and is completely unattached to the liner portion 310 in the open configuration shown in FIG. 12 and FIG. 13.

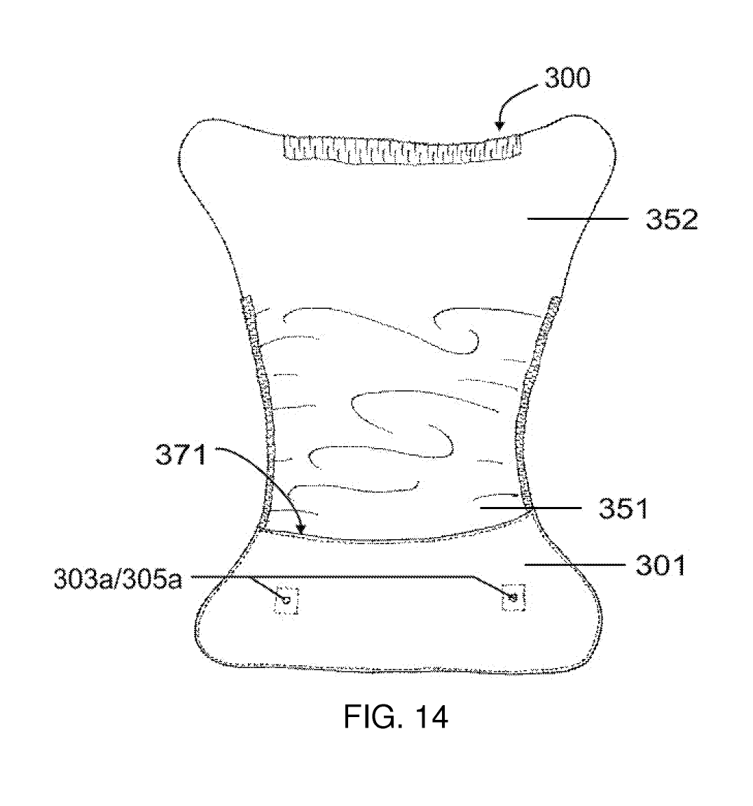

As shown in FIG. 14, the frontal barrier panel 301 may be attached to an inner surface of a frontal region 351 of the outer cover portion 300 to form a frontal pocket 371. The first fastener members 303a may be placed inside of the frontal pocket 371. The hole-openings 305a provide holes or openings through the frontal barrier panel 301 so that the first fastener members 303a may be reached and affixed through the hole-openings 305a.

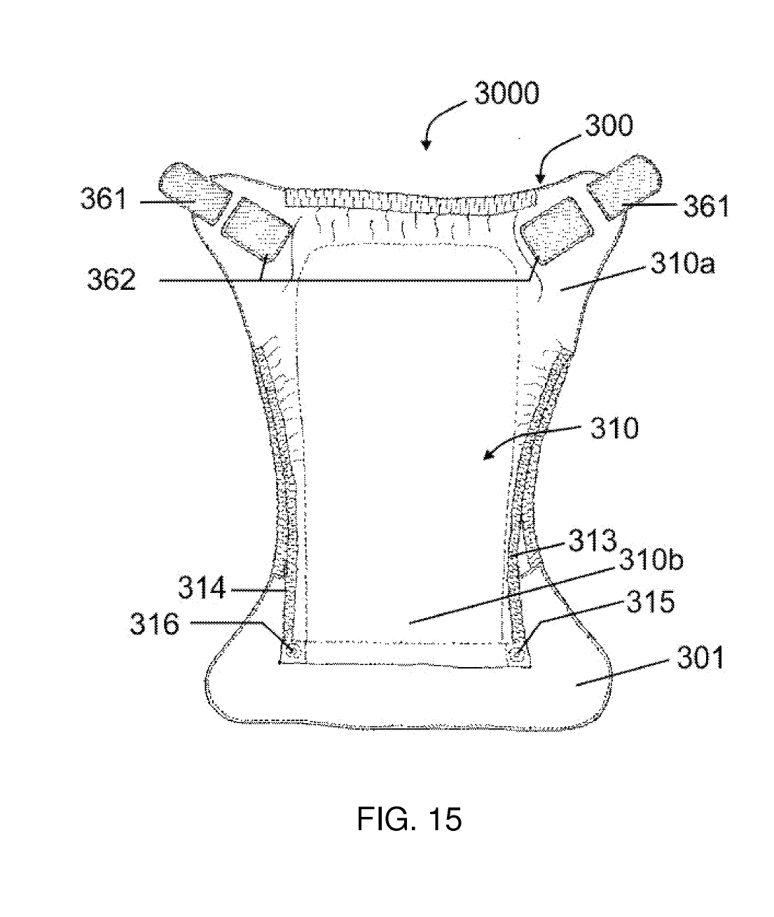

FIG. 15 is a top view of an inside of the outer cover portion 300 of the convertible reusable diaper 3000 with the liner portion 310 and an absorption portion 307 (shown in FIG. 11) semi-attached to the outer cover portion 300. The absorption portion 307 that comprises either an extended insert pad or a standard insert pad may be affixed to the liner portion 310 and placed between the liner portion 310 and the outer cover portion 300. A plurality of hook members 361 may be located at top corner areas of the attachable region 310a of the liner portion 310. The hook members 361 have matching loop members 362 so that the hook members 361 can be affixed to the loop members 362 when the hook members 361 are not in use. The detachable region 310b of the liner portion 310 is attached to the outer cover portion 300 via fastening the liner fastener members 315/316 to the frontal barrier panel 301. The liner portion 310 may also comprise the elastic disposed adjacent side edges 313 and 314 to provide a contoured fit around a wearer's body to dynamically prevent leakage.

The foregoing description and drawing merely explain and illustrate the invention, and the invention is not limited thereto, except as those skilled in the art who have the present disclosure before them will be able to make modifications and variations therein without departing from the scope of the invention.

What is claimed is:

1. A convertible reusable diaper comprising:
an outer cover portion including a frontal region, a crotch region, and a posterior region;
a barrier panel portion attachable to the outer cover portion;
at least one of a liner portion and an absorption portion;
one or more first fastener members each of which is attachable to at least one of the outer cover portion and the barrier panel portion;
one or more second fastener members each of which is attachable to at least one of the liner portion and the absorption portion, wherein at least one of the second fastener members is cooperatively engaged with at least one of the first fastener members, thereby allowing at least part of at least one of the liner portion and the absorption portion to be detachable to the outer cover portion; and
one or more hole-openings formed on the barrier panel portion, thereby allowing engagement between the first and the second fastener members.

2. The convertible reusable diaper of claim 1, wherein the barrier panel portion comprises:
at least one of a frontal barrier panel and a posterior barrier panel, the frontal barrier panel attachable to an inner surface of the frontal region of the outer cover portion, the posterior barrier panel attachable to an inner surface of the posterior region of the outer cover portion,
wherein the one or more first fastener members are attachable to at least one of the frontal barrier panel and the posterior barrier panel, and wherein the one or more hole-openings are formed on at least one of the frontal barrier panel and the posterior barrier panel.

3. The convertible reusable diaper of claim 2, wherein the one or more second fastener members comprises at least one liner fastener member attachable to the liner portion, wherein the at least one liner fastener member is engageable with at least one of the first fastener members through the corresponding hole-opening, thereby allowing the liner portion to be removable from and attachable to the outer cover portion.

4. The convertible reusable diaper of claim 2, wherein the liner portion comprises an attachable region and a detachable region, wherein the attachable region is attached to an inner surface of the outer cover portion, and wherein the detachable region is detached to an inner surface of the outer cover portion.

5. The convertible reusable diaper of claim 4, wherein the one or more second fastener members comprises at least one liner fastener member attachable to the detachable region of the liner portion, wherein the liner fastener member is engageable with at least one of the first fastener members through the corresponding hole-opening, thereby allowing the detachable region of the liner portion to be attachable to the outer cover portion.

6. The convertible reusable diaper of claim 2, wherein the one or more second fastener members comprises at least one absorption fastener member attached to the absorption portion, wherein the at least one absorption fastener member is engageable with at least one of the first fastener members through the corresponding hole-opening, thereby allowing the absorption portion to be removable from and attachable to the outer cover portion.

7. The convertible reusable diaper of claim 2, wherein the absorption portion comprises an attachable region and a detachable region, wherein the attachable region is attached to an inner surface of the outer cover portion, and wherein the detachable region is detached to an inner surface of the outer cover portion.

8. The convertible reusable diaper of claim 7, wherein the one or more second fastener members comprises at least one absorption fastener member each of which is attachable to the detachable region of the absorption portion, wherein the at least one absorption fastener members is engageable with at least one of the first fastener members through the corresponding hole-opening, thereby allowing the detachable region of the absorption portion to be attachable to the outer cover portion.

9. The convertible reusable diaper of claim 1, wherein the absorption portion is attachable with the liner portion.

10. The convertible reusable diaper of claim 1, wherein the liner portion is configured to be elasticized in a suitable manner to provide a contoured fit around a diaper wearer's body.

11. The convertible reusable diaper of claim 1, wherein at least one of the liner portion and absorption portion comprises at least one of polar fleece, fleece, micro fleece, suede cloth, linen, cotton, jute, hemp, bamboo, kenaf and sisal.

12. The convertible reusable diaper of claim 1, wherein at least one of the first and second fastener members comprises at least one of buttons, snaps, pins, adhesives, clasps, magnets, hooks and loop closures, elastic straps and adjustable straps.

13. The convertible reusable diaper of claim 1, wherein the liner portion comprises at least one of liquid-permeable material and natural material.

14. The convertible reusable diaper of claim 1, wherein the absorption portion comprises liquid absorbent material.

15. The convertible reusable diaper of claim 1, wherein at least one of the outer cover portion, the liner portion, the absorption portion, and the barrier panel portion are reusable, and are fabricated from washable materials.

16. The convertible reusable diaper of claim 1, wherein the outer cover portion further comprises an outer shell comprising at least one of polyester, fleece, wool and thermoplastic polyurethane laminated material.

17. The convertible reusable diaper of claim 1, wherein the barrier panel portion comprise at least one of polyester, fleece, wool, thermoplastic polyurethane laminated material, polar fleece, fleece, micro fleece, suede cloth, linen, cotton, jute, hemp, bamboo, kenaf and sisal.

18. The convertible reusable diaper of claim 1, wherein the frontal region of the outer cover portion comprises a plurality of adjustable fastener members to thereby allow size of the convertible reusable diaper adjustable.

19. The convertible reusable diaper of claim 1, wherein the posterior region of the outer cover portion comprises posterior adjustable fastener members at top corner areas, and wherein the frontal region of the outer cover portion comprises frontal adjustable fastener members engageable with corresponding posterior adjustable fastener members, whereupon positioning of the convertible diaper on a diaper wearer's body and allowing adjustable waist size of the convertible reusable diaper.

20. A convertible reusable diaper comprising:
an outer cover portion including a frontal region, a crotch region, and a posterior region;
a barrier panel portion attachable to the outer cover portion;
one or more first fastener members each of which is attachable to the barrier panel portion; and
one or more hole-openings formed on at least one of the barrier panel portion, thereby allowing engagement between the one or more first fastener members and one or more corresponding second fastener members, wherein each of the second fastener members is attachable to one of a liner portion and an absorption portion, and wherein at least one of the liner portion and the absorption portion is attachable to the outer cover portion.
